(19) United States
(12) Patent Application Publication
(10) Pub. No.: US 2010/0217130 A1
(43) Pub. Date: Aug. 26, 2010

(54) DEVICE FOR THE PRODUCTION OF PHOTOGRAPHIC IMAGES IN THE MOUTH REGION

(76) Inventor: Michael Weinlaender, Wien (AT)

Correspondence Address: KF ROSS PC, 5683 RIVERDALE AVENUE, SUITE 203 BOX 900, BRONX, NY, 10471-0900, US

(21) Appl. No.: 12/678,325

(22) PCT Filed: Sep. 15, 2008
(86) PCT No.: PCT/EP2008/007630

§ 371 Date: Mar. 22, 2010

(30) Foreign Application Priority Data
Sep. 21, 2007 (AT) A1489/2007
Mar. 13, 2008 (AT) A404/2008

(51) Int. Cl. A61B 6/00

(52) U.S. Cl. 600/476

(57) ABSTRACT

The invention relates to a device for the standardized and reproducible measurement and/or photography of the head, or of head regions, particularly of the intraoral region, characterized in that said device comprises—a cranial support (1) for the patient's head, and—an adjustable and positionable camera holder (2) for a camera (5), or a measuring device.

The invention concerns an apparatus for the standardized measurement and/or photography of the head, in particular of the intraoral area. In a plurality of medical working fields the doctor has the task of measuring and photographing certain areas of the face and in particular the oral areas. A particular task presents itself if such activities are to be standardized and reproducible and also, in particular, over longer periods of time. A typical example from the medical field is for example the aesthetic evaluation of the dentogingival complex with regard to dental implants.

With the present invention the object is attained of providing standardized and reproducible measurements and photographs of the face area and in particular of the intraoral area, with particular preference given to the dentogingival area. In particular the apparatus should enable the parameters of the patient position, that is to say of the head, the camera position and, if necessary, a mirror position, to be provided in a standardized and reproducible manner.

The apparatus and procedure proposed according to the invention are in particular then advantageous if variable processes proceed very slowly in the observed area. For example inflammatory processes with periodontal disease around the implants can proceed very slowly, and the tracking of such processes and also the documentation of these processes must proceed according to standardized and accurate criteria.

The present invention is primarily characterized in that an apparatus is provided that has a cranium restraint for the patient's head and an adjustable mount for a camera or a piece of measurement equipment. Another mount is also preferably provided for a mirror that can be introduced into the oral cavity of the patient, if exposures or measurements are to be undertaken in the posterior teeth area.

The apparatuses for the guidance of the camera and the mirror can be fixed in any position of the three-dimensional positioning system, preferably with quick release devices, and also reproducible by means of attached scales.

The reproducibility of the various head, camera and mirror positions is an important aspect.

The camera and mirror positioning units are two modules that are independent of one another. Thus for exposures in the so-called anterior teeth area, for example, the mirror module can be dispensed with.

Further advantageous embodiments of the invention can be found in the claims and in the following description and drawings.

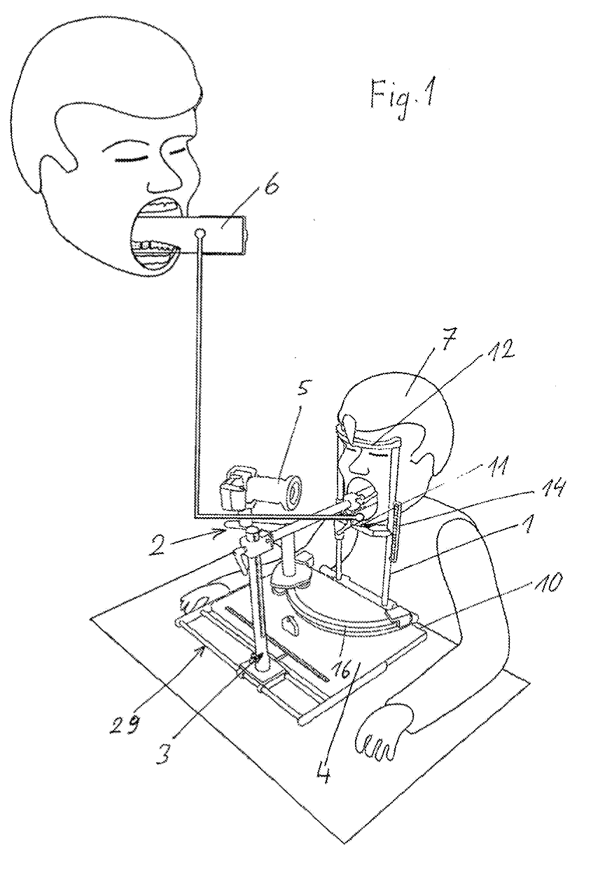

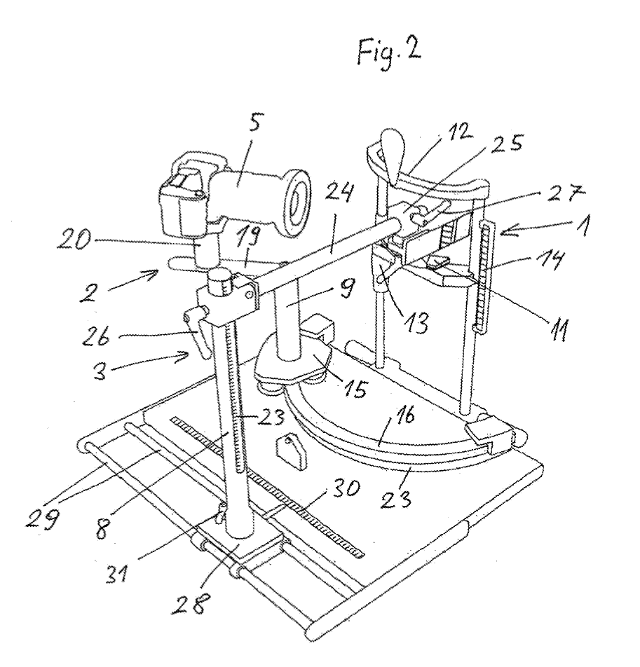

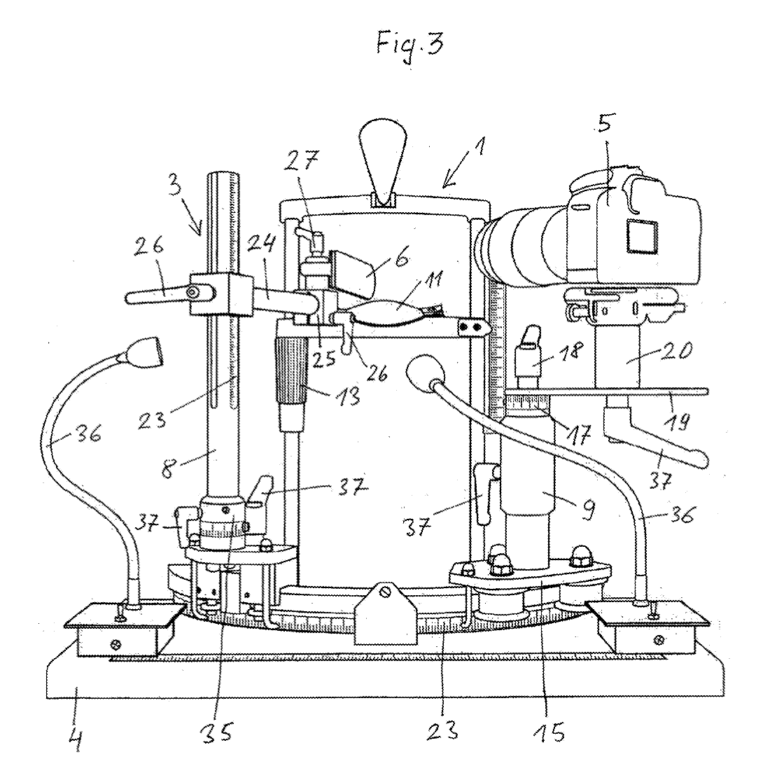

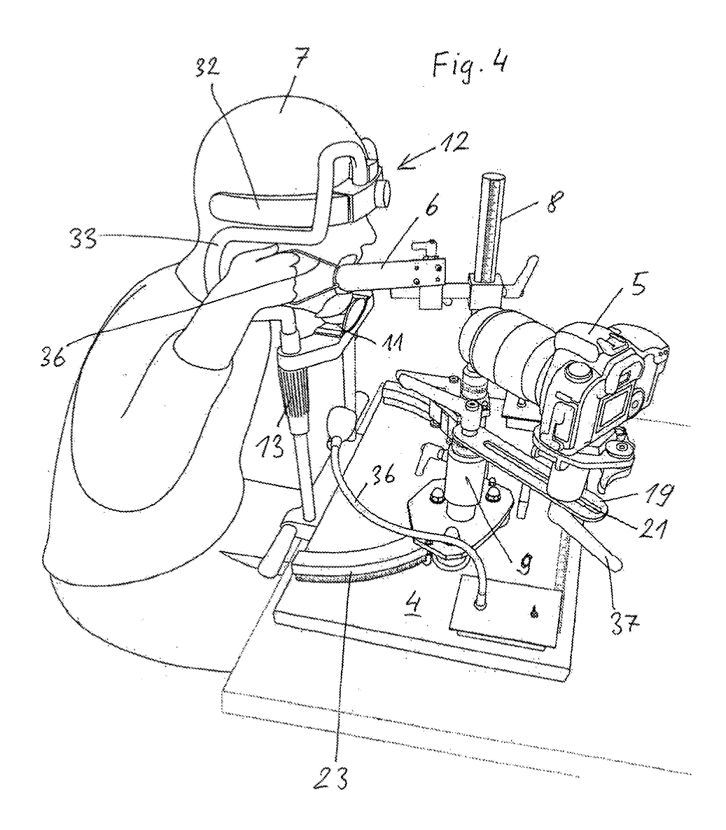

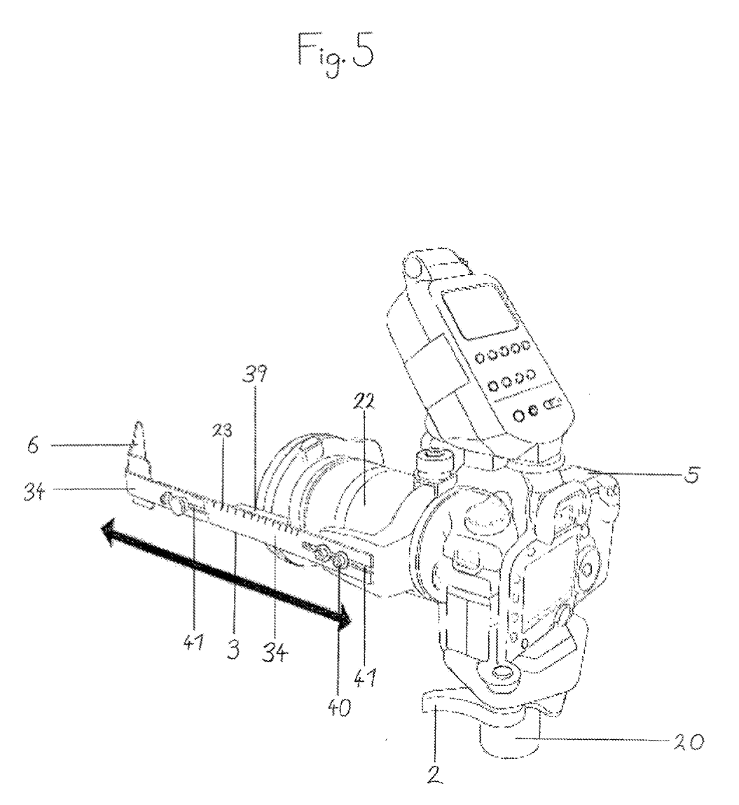

With the aid of three embodiments the invention is described in more detail. FIG. 1 shows the apparatus according to the invention in a first embodiment variant with a subject, while FIG. 2 shows the same apparatus in a somewhat enlarged oblique view. FIG. 3 is a front view of a second example of embodiment and FIG. 4 shows the apparatus according to FIG. 3 with a subject in place, wherein the head restraint is somewhat altered compared with that in FIG. 3. FIG. 5 shows a further embodiment of the mirror holder.

In the following description the words vertical and horizontal relate in each case to the normal operating position of the equipment. Movement directions relate consistently to the line of sight of the subject when in the investigation location.

The apparatus according to FIGS. 1 and 2 has a baseplate 4, to which all the other components of the apparatus are attached. With an appropriate weight the apparatus can simply be erected on a table, wherein preferably slip-proof elements such as for example rubber feet can improve its retention. Instead of the baseplate the parts of the apparatus can, however, also be directly connected to a table.

At the front edge 10 is located the cranium restraint 1 of the apparatus with a chin rest 11 and a cranium contact surface 12. By means of the adjusting screw 13 the chin rest 11 can be moved upward or downward, wherein a measurement scale 14 is provided at one edge of the cranium restraint 1, on which the adjusted height of the chin rest 11 can be read off and reproduced at any time.

For all examples of embodiment the cranium contact surface 12 is arranged for contact with the forehead. This contact surface can however also be related to other parts of the head.

As a component of the camera mount 2 a vertical column 9 is provided for the positioning of the camera 5, the column foot 15 of which can be moved on a curved guide rail 16. In the example represented the guide rail 16 is semicircular and the centre point of the circle is the point of intersection of the circle plane with a connecting vertical straight line that passes through the intraoral area of the subject. In this manner it is ensured that with a movement of the camera position along the curved guide rail the point of focus of the camera 5 always lies in the mouth area of the subject if the exposure area lies in the mouth area.

At the upper end of the vertical column 9 is arranged a horizontal retaining arm 19, to which the camera 5 is attached via a support tube 20.

As can be seen in FIG. 4, the retaining arm 19 can be provided with a lengthwise slot 21, so that the distance of the camera position from the exposure area can be altered.

Preferably this location along the slot 21 is also is graduated and can therefore be defined. The location of the column foot 15 along the guide rail 16 can be read off from a measurement scale 23.

Furthermore a graduated vertical column 8 is provided as a component of the mirror mount 3.

On the vertical column 8 is provided a moveable horizontal retaining arm 24, at the end of which a mount head 25 is arranged with the mirror 6. For rapid fixing locking levers 26, 27 are provided.

In the example of embodiment according to FIGS. 1 and 2 the mirror mount is supported with its column foot 28 that can move at right angles to the object of the exposure. For this purpose a mirror guide rail 29 arranged at right angles is provided, which in accordance with FIGS. 1 and 2 consists of two parallel bars. The position can be set and read off from the measurement scale 30. For fixing purposes a locking lever 31 is also provided here.

In the example of embodiment according to FIGS. 3 and 4 the curved guide rail 16 serves for attachment and guidance of both the camera mount 2 and also the mirror mount 3. The mirror guide rail and the camera guide rail are therefore a single rail with the corresponding measurement scale 23.

At the upper end of the vertical column 9 of the camera mount is located a graduated rotary head 17 and a locking lever 18, with which the horizontal arm can be established in a defined rotational orientation. The camera 5 sits on the retaining arm 19 via a support tube 20. Further locking levers are denoted by 37. The graduated vertical column 8 for the mirror mount sits on a graduated rotary knob 35.

The cranium contact surface 12 can be adapted according to FIG. 4 to the measurement conditions. In this example of embodiment a forehead contact surface is provided, which with lateral arms 32 spans the head laterally. The connecting bar 33 is shaped such that operation is facilitated. Thus the patient can manipulate the cheek holder 36 necessary for detaining the cheek in the correct position.

These cheek holders are so designed that the mirror is positioned intraorally into a recess of the cheek holder in the mouth of the patient. In this manner the cheek pressure of the patient does not have to be absorbed via the mirror and thus by the mirror positioning device, but rather can be equalized by the patient himself by means of the cheek holder that is held by him.

Furthermore the restraint of the cranium takes place via the chin rest 11. This can be individually adapted to the patient and by means of magnetic plates can easily be replaced.

For the standardized photographic exposure and measurement in the mouth area the patient assumes a sitting position and positions his chin in the chin rest 11 and the other appropriate part of his cranium on the cranium contact surface 12. Since the chin rest 11 can be adjusted according to the size of the patient, the position assumed can also be read off on the scale 14 of the head rest 1.

Subsequently the camera is brought into the correct position both in height and also in the correct location along the arc-shaped guide rail 16, wherein either the value can be read off and recorded from the measurement scale 23, or the camera is brought into this position along the measurement scale that had already been used for earlier exposures.

The mirror mount does not also have to be used in all cases, but is used very often. This is particularly the case if exposures are necessary in the posterior teeth area. Here the mirror can also fulfil a cheek holder function, or it can be combined with a cheek holder (see FIG. 4). The mirror can be three-dimensionally positioned in a few steps of manipulation, and the position can be read off. The mount head 25 can preferably be rotated about the tubular retaining arm 24 in a graduated manner, so that the rotational position of the mirror set can also be read off and reproduced.

FIG. 5 shows an advantages variant of the apparatus according to the invention where the holder 3 carrying the mirror 6 is secured to the camera 5 or on the mirror mount 2. With this arrangement of the mirror mount the above-described vertical column for the mirror and the associated parts such as the mirror guide rail 29 are not provided. The device can also be set up as shown in FIGS. 1 to 4 such that actually the mirror mount according to FIG. 5 is used. In any case the parts of the apparatus for holding the cranium and the mirror mount are according to FIGS. 1 to 4.

In FIG. 5 the mirror mount 3 is mounted on the objective 22 of the camera 5. The mirror mount 3 is of adjustable length with two relatively shiftable mount parts 34 that can be locked together by screws 40. The screws 40 are guided in elongated slots 41 of the mount parts 34. In order to determine the spacing of the mirror 6 from the objective a scale 23 is provided on one of the surfaces of the mirror mount 3, in this embodiment the scale being on the outer surface of the mirror mount 3. This way the spacing between the mirror and the objective can be set and later reproduced. In order to stabilize the holder the mirror mount4 3 has a brace 30 bearing on the outer objective ring 38 of the camera.

The advantage of this variant of the invention is that the number of parts needed for standardizing is reduced. The mirror is positioned with the camera, making operation easier.

### REFERENCE SYMBOL LIST

01. Cranium restraint 26. Locking lever
02. Camera mount 27. Locking lever
03. Mirror mount 28. Column foot
04. Baseplate 29. Mirror guide rail
05. Camera 30. Measurement scale
06. Mirror 31. Locking lever
07. Head 32. Arm
08. Mirror vertical column 33. Connecting bar
09. Camera vertical column 34. Mount part
10. Front edge 35. Rotary head
11. Chin rest 36. Cheek holder
12. Cranium contact surface 37. Other locking levers
13. Adjusting screw 38. Objective ring
14. Measurement scale 39. Brace
15. Column foot 40. Screw
16. Camera guide rail 41. Slot
17. Rotary head
18. Locking lever
19. Camera retaining arm
20. Support tube
21. Slot
22. Objective
23. Measurement scale
24. Mirror retaining arm
25. Mount head

1. An apparatus for the standardized and reproducible measurement or photography of the head or of the intraoral area, the apparatus comprising:
a cranium restraint for the head of the patient, and
a mount that can be adjusted and set for a camera or a piece of measuring equipment.

2. The apparatus according to claim 1, further comprising
a mirror mount for a mirror that can be introduced into the oral cavity of the patient.

3. The apparatus according to claim 1, wherein the cranium restraint for the head of the patient can be adjusted in height.

4. The apparatus according to claim 1 wherein a device is provided to indicate the head location of the patient.

5. The apparatus according to claim 1 wherein the camera mount is provided with a positioning device.

6. The apparatus according to claim 1 wherein a display is provided indicating the orientation of the camera.

7. The apparatus according to claim 1 wherein the mirror mount is provided with a positioning device for the mirror.

8. The apparatus according to claim 1 wherein a display is provided indicating the orientation of the mirror.

9. The apparatus according to claim wherein the camera mount can be positioned along an arc-shaped camera guide rail.

10. The apparatus according to claim 9, wherein the camera guide rail is circular and the center point of the circle is arranged such that the camera can swivel about a point in the oral cavity.

11. The apparatus according to claim 1 wherein the distance from the camera to the head and the angular position of the camera can be adjusted.

12. The apparatus according to claim 1 wherein the mirror mount is independent of the camera mount and can be moved at right angles to the head.

13. The apparatus according to claim 1 wherein the mirror mount can be adjusted on a mirror guide rail arranged at right angles.

14. The apparatus according to claim 1 wherein the mirror mount and the camera mount can be arranged on the same camera guide rail.

15. The apparatus according to claim 1 wherein the guide rails for the mirror mount and the camera mount and also the cranium restraint are attached to a baseplate.

16. The apparatus according to one claim 1 wherein the camera and the mirror can be brought into a defined three-dimensional orientation independently of one another.

17. The apparatus according to claim 1 wherein for the height adjustment of the camera and the mirror graduated vertical columns are provided, on which retaining arms are arranged that carry the mirror or the camera.

18. The apparatus according to claim 1 wherein the vertical columns or the retaining arms can be rotated by graduated rotary heads.

19. The apparatus according to claim 1 wherein the cranium restraint has a chin rest and a forehead contact surface.

20. The apparatus according to claim 1 wherein illumination means is provided for illuminating the object of the exposure.

21. The apparatus according to claim 1 wherein the cranium restraint has a cranium contact surface lying against the forehead, on which extending arms are arranged to both sides of the head.

22. The apparatus according to claim 21, wherein a connecting bar is provided that extends on both sides of the head to the cranium contact surface, wherein the connecting bar is curved to release the cheek area of the head.

23. (canceled)

24. The apparatus according to claim 1 wherein the chin rest is attached by a magnet to the cranium restraint.

25. The apparatus according to claim 1, wherein the mount carrying the mirror is fixed or fixable on the camera or on the camera mount.

26. The apparatus according to claim 25, wherein the mirror mount is fixable on the objective of the camera.

27. The apparatus according to claim 25 wherein the mirror mount is of adjustable length.

28. The apparatus according to claim 27, wherein the to adjust the length of the mirror mount there are at lest two relative shiftable and fixable mount parts and that to set the spacing between the mirror and the objective a scale is provided.

29. The apparatus according to claim 25 wherein the mirror mount has a brace bearing on an outer objective ring of the camera.
